(19) United States
(12) Patent Application Publication
(10) Pub. No.: US 2005/0059945 A1
(43) Pub. Date: Mar. 17, 2005

(54) Disposable clothing

(76) Inventor: Nancy Lee Schulson, Chicago, IL (US)

Correspondence Address: Harold V Stotland;Seyfarth Shaw, 42nd Floor, 55 East Monroe Street, Chicago, IL, 60603-5803, US

(21) Appl. No.: 10/626,946

(22) Filed: Jul. 25, 2003

(51) Int. Cl. A61F 13/15; A61F 13/20

(52) U.S. Cl. 604/385.24

(57) ABSTRACT

Disposable garments, such as t-shirts, shorts, pants and the like are formed from nonwoven fabric and are seamed with elastomeric material which absorbs the stresses created when a wearer undergoes physical activity such as exercise. Absorbent material is used to line the garments to collect perspiration.

### BACKGROUND OF THE INVENTION

This invention is concerned generally with articles of clothing and, more specifically, to clothing made to be sturdy, absorbent and disposable for use as exercise or workout clothing.

People using exercise facilities such as gyms and fitness centers typically carry with them exercise-appropriate clothing to use during workouts. After exercising soiled and sweaty exercise clothing is either carried home to be laundered or, in some instances, can be laundered by the workout facility and returned to the user's locker or other storage area. Because of such factors as laundering cost, initial purchase price, sanitation issues and the wide range of sizes and garment preferences, workout centers do not usually stock a supply of workout clothes for those who do not bring their own with them. Fitness centers in facilities such as hotels and the like deal with this problem on a more frequent basis with travelers or guests who do not bring clothing with them but want to use the facilities during their stay. Those guests that do remember to bring their own clothing are also faced with the task of either laundering it before returning home or packing damp, used clothing in with their other personal effects.

The concept of disposable and, in some cases, absorbent and disposable items of clothing is well represented in the prior art.

U.S. Pat. No. 6,049,909 (Anderson) teaches and describes a disposable infant garment made from breathable paper impregnated with a waterproofing agent. Anderson discloses and claims a garment made to be worn the same as conventionally-made children's playwear, yet intended to be disposable. As claimed, the invention resides in the material from which the garment is made and the manner in which the clothing blank is folded and fastened together to form the garment.

U.S. Pat. No. 5,915,532 (Williams) teaches and describes a disposable headband formed from an outer, non-woven sheet surrounding an insert made from a permeable material containing an absorbent material in the form of potassium or sodium polyacrylate crystals. The crystals absorb perspiration through the permeable layer. Williams also teaches the use of a label attached to the headband.

U.S. Pat. No. 5,770,529 (Dennis, et al.) teaches and describes a liquid-distribution garment to be used as a liner to be worn under impermeable protective clothing. Dennis, et al. claims not only a specific construction but specific wicking rates for the fabric sections of the garment. While disposability is not claimed as a feature of the invention, the patent does state that the garment is intended to be inexpensive enough to be disposable.

U.S. Pat. No. 5,103,501 (Meisels) teaches and describes traveling and disposable underwear featuring absorbent pads placed at those points in the garment where needed. Meisels also claims the use of elastic bands and magnetic fasteners to make the garment adjustable to various sizes.

U.S. Pat. No. 5,058,210 (Tivis) teaches and describes a disposable sweat liner for safety hats consisting of three layers of absorbent padding shaped to fit within a protective helmet and to cover the forehead and the crown of the user's head. Tivis specifically claims the liner to be disposable and further claims a fastening system adapted to attach the liner to the inner support web of the helmet.

U.S. Pat. No. 4,947,867 (Keeton) teaches and describes surgical clothing and labeling means therefor. Keeton's invention is a disposable surgical gown to be worn by a patient into surgery and which also included labels attachable to the gown and to the patient's body to alert the surgical team to the patient's identity and the surgical procedure to be performed, as shown in FIG. 1 of the patent.

U.S. Pat. No. 4,819,275 (Lunt) teaches and describes a disposable gown for medical patients consisting of a blank formed of non-woven fabric and shaped to fit onto and be secured to a patient's torso.

U.S. Pat. No. 3,911,499 (Benevento, et al.) teaches and describes a disposable medical gown made without seams to form a bacteria barrier.

U.S. Pat. No. 3,720,957 (Patience) teaches and describes a conformable disposable garment, sections of which are formed with micropleats, as seen in FIG. 5 of the patent, to allow freedom of movement and adjustable fit even though the gown fabric is inelastic.

U.S. Pat. No. 3,451,062 (Bradley) teaches and describes a disposable examination gown made from a single sheet of material and attachable with peel-out ties formed as part of the garment.

U.S. Pat. No. 5,601,542 (Melius, et al.) teaches and describes an absorbent composite included as part of a disposable absorbent garment. The absorbent material is held between layers of semi-permeable, non-woven material.

U.S. Pat. No. 5,147,343 (Kellenberger) teaches and describes absorbent products containing hydrogels with ability to swell against pressure where the superabsorbent material is dispersed among a matrix of fibers sandwiched between a liquid-impermeable backing material and a liquid-permeable facing material.

U.S. Pat. No. 5,622,581 (Ducker, et al.) teaches and describes a disposable garment with de-elasticized elastic members and method for making same for use with garments having elastic gathers and then either chemically or mechanically de-elasticizing portions of the elastic gathers in order to vary the tension with which the gathers grip the wearer.

U.S. Pat. No. 5,490,846 (Ellis, et al.) teaches and describes a surge management fibrous nonwoven web for personal care absorbent articles and the like consisting of a fibrous, nonwoven web used to absorb fluids very rapidly and distribute the fluids to an absorbent material.

U.S. Pat. No. 4,641,381 (Heran, et al.) teaches and describes disposable underpants, such as infant's training pants and the like using disposable material formed into a pair of training pants having an elasticized waist and elasticized leg openings having a liner consisting of an absorbent batt covered with layer of nonwoven material that has an inner layer of plastic material and an outer layer of nonwoven fibrous material.

U.S. Pat. No. 4,610,681 (Strohbeen, et al.) teaches and describes disposable underpants having discrete outer seals with the garment having elasticized waist and leg openings made with seams sealed with a plurality of narrow lines preferably formed with an ultrasonic anvil.

U.S. Pat. No. 5,429,629 (Latimer, et al.) teaches and describes an absorbent structure having improved fluid surge management and product incorporating same comprising a garment having a “surge management” portion for rapidly receiving and dispersing liquids and a retention portion to desorb fluids form the surge management portion and to store the fluids.

U.S. Pat. No. 6,240,563 (Niedermeyer) teaches and describes apparel with panel attachments along selected margins. Niedermeyer is concerned with construction techniques for manufacturing clothing from the types of non-woven fabrics used to make disposable and reusable garments, and teaches the use of bonded seams to keep the garment together.

U.S. Pat. No. 5,553,326 (Moore) teaches and describes a removable, washable sweatband for hats using a “zip-loc” type fastener to hold the band in place within the hat.

U.S. Pat. No. 5,098,419 (Gold) teaches and describes an undergarment to be worn by incontinent persons, and is exemplary of clothing using fabric to form an impervious barrier together with absorbent fabric.

United States patent (Dillon) teaches and describes a unisex shirt and methods for making same and describes the use of the garment as a surgical scrub.

While certain of these references disclose aspects of clothing that would be desirable in disposable workout wear, no reference discloses constructions suited for the uses to which such clothing is typically put.

Accordingly, it is an object of the present invention to provide workout clothing that is sufficiently sturdy in construction to stand up to the stresses created during exercise movements.

It is a further object of the present invention to provide such clothing with elasticized seam areas sufficiently stressable to allow the clothing to be made from generally lightweight material yet have the strength to be used for exercise.

It is a further object to provide such clothing in forms which use inexpensive materials for construction and are simple and inexpensive to manufacture.

Yet another object is for such clothing to be provided in forms which are disposable and can be recycled.

Still another object is for such clothing to be easily personalized with initials, logos and the like.

### BRIEF DESCRIPTION OF THE INVENTION

A construction for disposable garments intended for use as exercise clothing combines the use of non-woven fabrics, absorbent material layers and elastic, expandable seaming at major stress points. Such constructions are used to make conventional and familiar workout items such as sleeved and sleeveless T-shirts, shorts, headbands and the like. The non-woven fabrics are selected to be lightweight, attractive, sturdy and capable of being marked or imprinted with logos, graphics and the like. The placement of expandable seam constructions on the garments allows the garments to absorb the stresses created by exercising yet keep the garments inexpensive to make and comfortable to wear.

While the following describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described. Where means-plus-function clauses are used such language is intended to cover the structures described herein as performing the recited functions and not only structural equivalents but equivalent structures as well. For the purposes of the present disclosure, two structures that perform the same function within an environment described above may be equivalent structures.

### BRIEF DESCRIPTION OF THE DRAWINGS

These and further objects of the present invention will become more apparent upon consideration of the accompanying drawings, wherein:

FIG. 1 is a front elevational view of a T-shirt constructed in accordance with a preferred embodiment of the present invention;

FIG. 2 is a front elevational view of a sleeveless T-shirt constructed in accordance with a preferred embodiment of the present invention;

FIG. 3 is detail of the underarm stitching of the embodiment of FIG. 1;

FIG. 4 is a detail of the collar stitching of the embodiment of FIG. 1;

FIG. 5 is a front elevational view of a front shirt panel;

FIG. 6 is a front elevational view of a rear shirt panel;

FIG. 7 is a front elevational view of a pair of exercise pants embodying the teachings of the present invention; and

FIG. 8 is a side sectional view of a second embodiment of the clothing material.

### DETAILED DESCRIPTION OF THE INVENTION

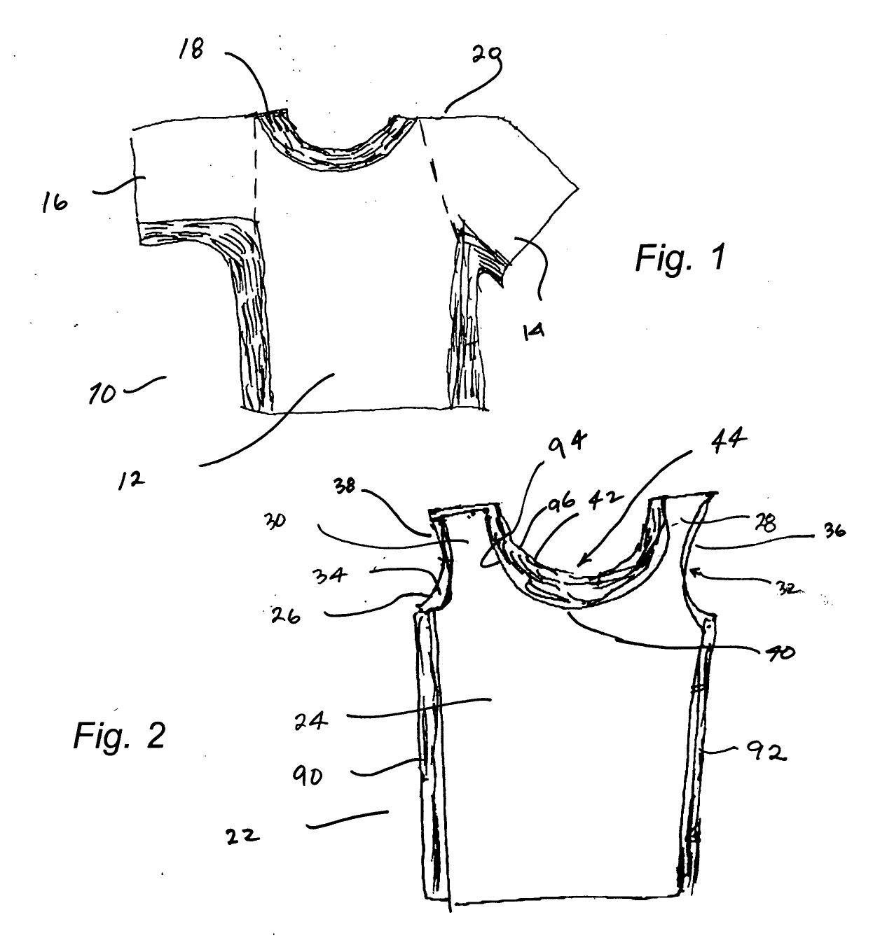

For the purposes of describing the invention, the articles of clothing hereinafter described are constructed in much the same manner as their more conventional counterparts. For example, a sleeved T-shirt such as that shown in FIG. 1 is typically assembled from a front panel, a back panel, sewn-in sleeves and a sewn-in neck band or collar.

Referring now to FIG. 1 the numeral 10 identifies generally a sleeved T-shirt constructed in accordance with a preferred embodiment of the invention.

Shirt 10 has a front panel 12, a left sleeve 14, a right sleeve 16, a collar 18 and a rear panel 20, not shown but understood to be substantially similar in dimension and shape to front panel 12.

Referring to FIG. 2 a second version of an exercise shirt is shown as a sleeveless shirt 22 having a front panel 24 and a rear panel 26. Front panel 24 has a left shoulder “strap” 28 and a right strap 30, while rear panel 26 has a corresponding left strap 32 and a right strap 34. As described hereinafter, left straps 28, 32 and right straps 30, 34 are seamed together to form, respectively, left arm hole 36 and right arm hole 38. Front panel 24 also has a front collar segment 40 while rear panel 26 has a corresponding rear collar segment 42 which, when joined as described below, combine to form a neck opening 44.

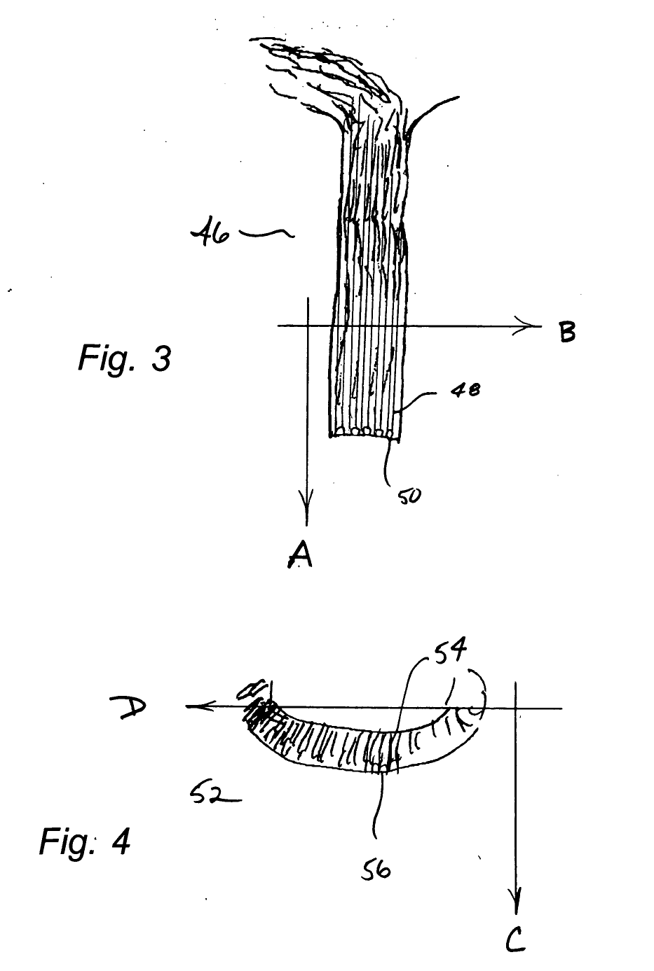

Referring now to FIG. 3, a first segment of elasticized seaming 46 is shown, consisting of a number of parallel seams 48 extending longitudinally along segment 46 to form a series of parallel channels 50. When stressed longitudinally in direction A segment 46 can stretch to a certain limit but can stretch to a significantly greater degree when stressed laterally in direction B.

Referring now to FIG. 4 a second segment of elasticized seaming 52 is shown, adapted to be used as collar segment 18, 40 or 42. Collar seams 54 are sewn across the shortest dimension of segment 52, forming transverse channels 56. Segment 52 thus stretches to a greater degree in direction D than in direction C.

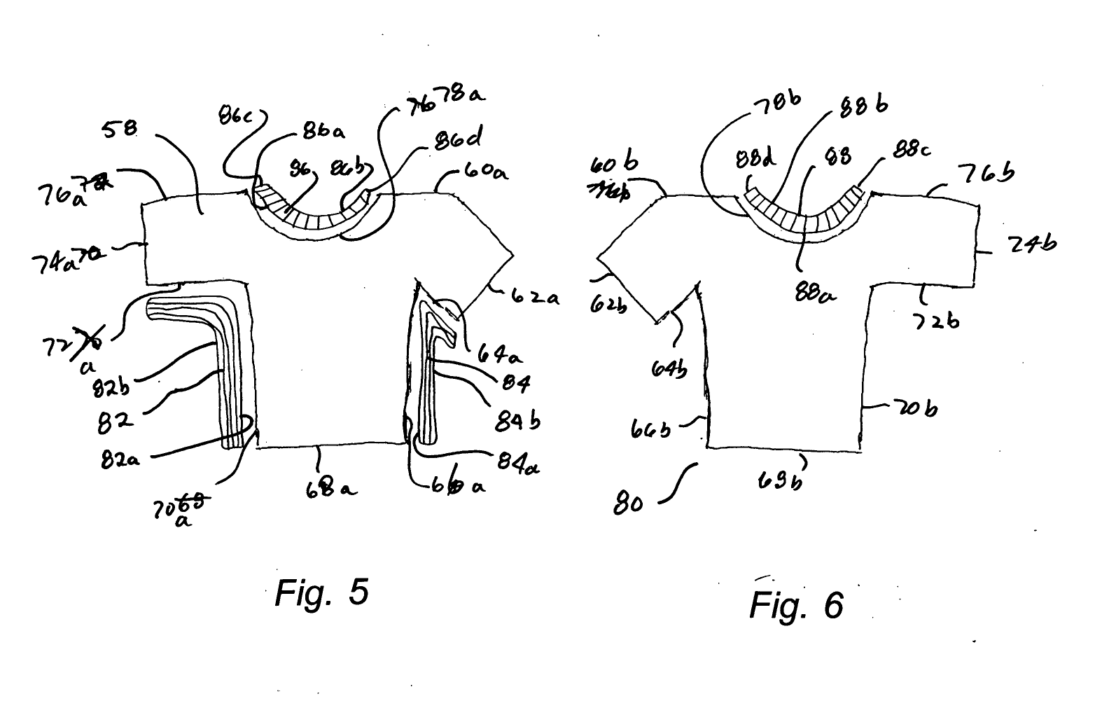

Referring now to FIGS. 5 and 6 the present invention may be described in full detail. In FIG. 5 a t-shirt front panel 58 is shown, preferably formed from a non-woven fabric such as polypropylene fibers. Panel 58 has a first shoulder edge 60a, a first sleeve edge 62a, a first arm edge 64a, a first torso edge 66a, a bottom edge 68a, a second torso edge 70a, a second sleeve edge 72a, a second arm edge 74a, a second shoulder edge 76a and a collar edge 78a, said edges defining the shape of panel 58.

Similarly, in FIG. 6 a t-shirt rear panel 80 is shown, having a first shoulder edge 60b, a first sleeve edge 62b, a first arm edge 64b, a first torso edge 66b, a bottom edge 68b, a second torso edge 70b, a second sleeve edge 72b, a second arm edge 74b, a second shoulder edge 76b and a collar edge 80b, said edges defining the shape of rear panel 80.

A first elastic segment 82 is cut such that the length of segment 82 is equal to the combined lengths of edges 70a, 72a. Segment edge 82a is then sewn to edges 70a, 72a. In like fashion, a second elastic segment 84 is cut such that the length of segment 84 is equal to the lengths of edges 64a, 66a and is then sewn to edges 64a, 66a. Segments 82, 84 are constructed as segment 46 is described and as shown in FIG. 3.

A first elastic collar segment 86 is positioned at and is cut to the length of collar edge 78a. Collar segment edge 86a is then sewn to edge 78a, with collar edges 86c, 86d terminating proximate edges 76a, 60a respectively.

In similar fashion. a second elastic collar segment 88 is positioned at and is cut to the length of collar edge 78b. Collar segment edge 88a is then sewn to edge 78b, with collar edges 86c, 86d terminating proximate edges 76b, 60b respectively.

Collar segments 86, 88 are constructed as described and as shown in FIG. 4.

Form 58 is then sewn to form 80 in the following fashion: edge 60a sewn to edge 60b, edge 76a sewn to edge 76b, edge 82b sewn to edge 70b, edge 84b sewn to edge 66b and, to complete collar 18 of FIG. 1, edges 86c and 88c are sewn together, as are edges 86d and 88d.

In similar fashion, shirt 22 shown in FIG. 2 is assembled such that front and rear panels 24, 26 are joined together at one side by elastic segment 90 and at the opposite side by elastic segment 92, with segments 90, 92 constructed as described in connection with FIG. 3. Elastic collar segments 94, 96, constructed as described and as shown in FIG. 4 are sewn to form collar opening 44.

So constructed, shirt 10 provides sufficient stretch capability to enable panels 58, 80 to be made from relatively inelastic non-woven material and still provide the freedom of motion and comfort to allow shirt 10 to be used over the ranges of motion necessary for physical exercise.

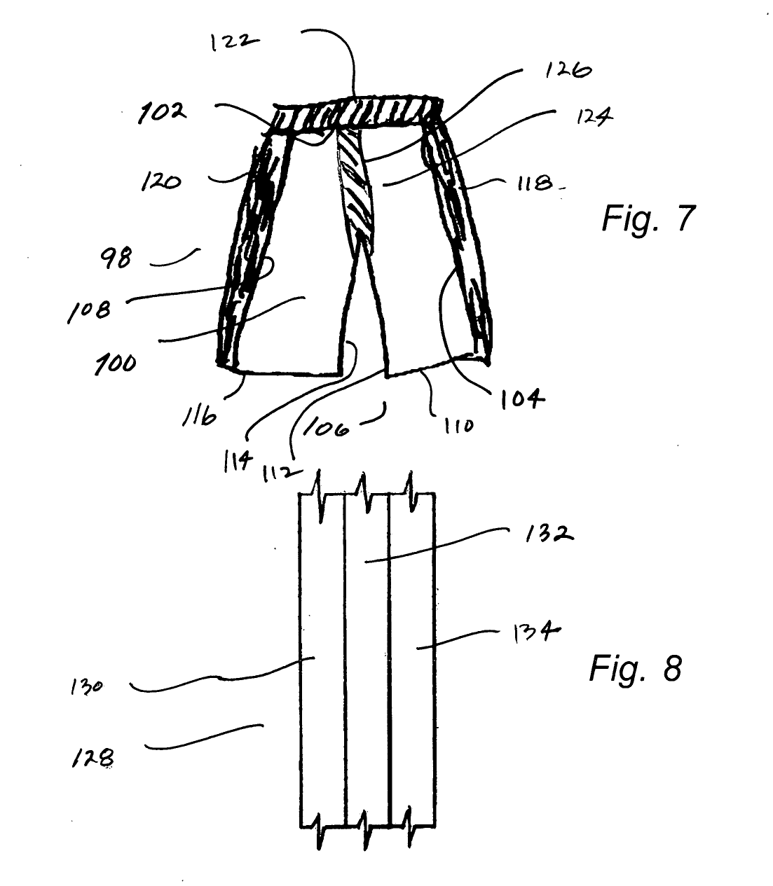

As seen in FIG. 7, the same construction techniques and materials can be used to make exercise shorts and pants. In FIG. 7, a first embodiment of a pair of exercise shorts 98 has a front panel 100 having a top edge 102, a left side edge 104, a bottom edge 106 and a right side edge 108. Bottom edge 106 has a left segment 110, a left inseam edge 112, a right inseam edge 114 and a right segment 116. In the embodiment shown a rear panel (not shown) is a mirror image of front panel 100.

To construct shorts 98 in accordance with the present invention, a left side seam 118 made from longitudinal elastic segments as described above is attached to left side edge 104 of the front panel 100 and the corresponding edge on the rear panel. Similarly, a right side seam 120 is formed from an elasticized segment and is attached to right side edge 108 and its corresponding rear panel edge. Left inseam edge 112 is likewise seamed to its rear panel counterpart, as is right inseam edge 114. An elastic waistband 118 is formed from a transverse elastic segment as described above.

In a second embodiment, a crotch seam 124 is formed by cutting panel 100 from the point at which left and right inseam edges 112, 114 meet to top edge 102 and using an elasticized segment 126 to attach the panel edges together, it beign understood that the same modifications are made to the mirror-image rear panel.

When manufacturing pants instead of shorts, edges 104, 108, 112 and 114 are lengthened to the desired pants size genrally following the same construction techniques as described above.

The direction in which seams 118, 120 and 126, and waistband 122 stretch can advantageously be set by using elastomeric materials such as those made under the trademark FLEXCEL™ by Kimberly-Clark Corporation.

As seen in FIG. 8, the fabric panels used to assemble the embodiments above can be made with several plies, designed to absorb and retain perspiration during workouts. As seen in cross-section in FIG. 8, panel 128 has a first, outer layer 130 formed from non-woven material. An inner layer 132 formed from a hydrophilic or absorbent material such as that made under the trademark ABSORB.LOC by Kimberly-Clark Corporation can be laminated to layer 130. If desired, an inner layer 134 can be added to form a three-ply construction, with layer 134 formed from a semi-permeable material such as commercially available spunbonded or carded fibrous webs as described in U.S. Pat. No. 5,147,343, or polyester or polypropylene fibers as described in U.S. Pat. No. 5,429,629, to allow moisture to travel through layer 134 to be trapped in layer 132 without soaking through layer 110.

Clothing such as described above can be provided by exercise facilities for one-time use and then discarded and recycled, reducing laundry costs and the inconvenience of traveling with clean and (ultimately) soiled workout clothes. The surface of the non-woven material can be printed or written upon and names, designs, club logos, sizes and even advertisements readily displayed. Facilities such as schools, rehabilitation and senior centers and hotels will find use for the present invention for disposable exercise or emergency garments. Runners can find particular use for the present invention for garments that can be worn at the start of a long-distance race such as a marathon and discarded along the race route as the runner's temperature goes up. The same garments can be used to warm race participants at the end of a race held in cold weather.

1. A disposable garment made from a number of individual panels, said garment comprising:
at least two individual panels which, when attached together, form said garment,
each said panel formed from non-woven material,
each said panel having one or more panel edges which, when joined to corresponding edges on other of said panels form said garment;
means for joining said panel edges one to the other,
said joining means comprising elasticized material attached to one said edge on one said panel and to one said corresponding edge on another said panel,
said elasticized material made to stretch in a direction of major stress when said garment is worn whereby said panels are not stretched.

2. The construction as recited in claim 1 wherein said non-woven material is formed from polypropylene fibers.

3. The construction as recited in claim 1 wherein said elasticized material is formed from non-woven elastomeric meltblown fabric.

4. The construction as recited in claim 1 wherein said elasticized material is a stretch bonded laminate formed from non-woven elastomeric meltblown fabric.

5. The construction as recited in claim 1 wherein said panels include a layer formed from non-woven absorbent material.

6. The construction as recited in claim 5 wherein said absorbent material is a hydrophilic fiber.

7. The construction as recited in claim 5 wherein said panels include an inner layer of semipermeable material covering said absorbent layer.

8. The construction as recited in claim 7 wherein said semipermeable material is formed from non-woven polymeric material.

9. The construction as recited in claim 8 wherein said non-woven material is selected from polypropylene or polyethylene fibers.

10. A disposable shirt or shirt-like garment, said garment comprising:
a first shirt panel having a left side edge, a right side edge, a top edge and a bottom edge;
a second shirt panel having a left side edge, a right side edge, a top edge and a bottom edge,
said first and second shirt panels formed from non-woven material;
means for joining selected of said panel edges one to the other,
said joining means comprising elasticized material attached to one said selected edge on said first shirt panel and to one said selected edge on said second shirt panel,
said elasticized material made to stretch in a direction of major stress when said garment is worn.

11. The apparatus as recited in claim 10 wherein said shirt is formed by joining selected portions of said first and second panel top edges one to the other to form a top seam, and
using said joining means to attach selected portions of said first and second panel left side edges one to the other to form a left seam, and selected portions of said first and second panel right side edges one to the other to form a right seam,
said left seam and said top seam defining therebetween a left arm opening,
said right seam and said top seam defining therebetween a right arm opening,
said attached portions of said top seam defining a neck opening.

12. The apparatus as recited in claim 11 wherein said joining means is made to stretch in a direction generally perpendicular to said left and right seams to allow said first and second panels to move apart one from the other when one or both of said left and right seams are stressed.

13. The apparatus as recited in claim 11 wherein said top seam is formed by using said joining means to attach selected portions of said first and second top edges together one to the other.

14. The apparatus as recited in claim 10 wherein said non-woven material is formed from polypropylene fibers.

15. The construction as recited in claim 10 wherein said elasticized material is formed from non-woven elastomeric meltblown fabric.

16. The construction as recited in claim 10 wherein said elasticized material is a stretch bonded laminate formed from non-woven elastomeric meltblown fabric.

17. The construction as recited in claim 10 wherein said panels include a layer formed from non-woven absorbent material.

18. The construction as recited in claim 17 wherein said absorbent material is a hydrophilic fiber.

19. The construction as recited in claim 17 wherein said panels include an inner layer of semipermeable material covering said absorbent layer.

20. The construction as recited in claim 19 wherein said semipermeable material is formed from non-woven polymeric material.

21. The construction as recited in claim 20 wherein said non-woven material is selected from polypropylene or polyethylene fibers.

22. A disposable pants or pant-like garment, said garment comprising:
a first panel having a left side edge, a right side edge, a top edge and a bottom edge,
said bottom edge having a left segment generally perpendicular to said left side edge, a first inseam segment extending from said leg segment, a second inseam segment extending from said first inseam segment and a right segment extending from said second inseam segment to and generally perpendicular to said right edge;
a second panel having a left side edge, a right side edge, a top edge and a bottom edge,
said bottom edge having a left segment generally perpendicular to said left side edge, a first inseam segment extending from said leg segment, a second inseam segment extending from said first inseam segment and a right segment extending from said second inseam segment to and generally perpendicular to said right edge;
said first and second panels formed from non-woven material;
means for joining selected of said panel edges one to the other,
said joining means comprising elasticized material attached to one said selected edge on said first panel and to one said selected edge on said second panel,
said elasticized material made to stretch in a direction of major stress when said garment is worn.

23. The apparatus as recited in claim 22 wherein said garment is formed by joining said first and second left inseam segments one to the other to form a left inseam,
joining said first and second right inseam segments one to the other to form a right inseam, and
using said joining means to attach selected portions of said first and second panel left side edges one to the other to form a left leg seam, and selected portions of said first and second panel right side edges one to the other to form a right leg seam,
said left leg seam and said left inseam defining therebetween a left leg opening,
said right leg seam and said right inseam defining therebetween a right leg opening,
said unseamed top edge defining a top opening extending from said left leg seam to said right leg seam.

24. The apparatus as recited in claim 23 wherein said joining means is made to stretch in a direction generally perpendicular to said left and right leg seams to allow said first and second panels to move apart one from the other when one or both of said left and right seams are stressed.

25. The apparatus as recited in claim 23 wherein said left and right inseams are formed by using said joining means to attach said first and second left inseams together and said first and second right inseams together.

26. The apparatus as recited in claim 22 wherein said non-woven material is formed from polypropylene fibers.

27. The construction as recited in claim 22 wherein said elasticized material is formed from non-woven elastomeric meltblown fabric.

28. The construction as recited in claim 22 wherein said elasticized material is a stretch bonded laminate formed from non-woven elastomeric meltblown fabric.

29. The construction as recited in claim 22 wherein said panels include a layer formed from non-woven absorbent material.

30. The construction as recited in claim 29 wherein said absorbent material is a hydrophilic fiber.

31. The construction as recited in claim 29 wherein said panels include an inner layer of semipermeable material covering said absorbent layer.

32. The construction as recited in claim 31 wherein said semipermeable material is formed from non-woven polymeric material.

33. The construction as recited in claim 32 wherein said non-woven material is selected from polypropylene or polyethylene fibers.

34. The apparatus as recited in claim 23 wherein said garment included a crotch seam,
said crotch seam formed from said top edge of said first panel to that point at which said first and second first panel inseam segments meet, and from said top edge of second panel to that point at which said first and second panel inseam segments meet,
at least a portion of said crotch seam formed by attaching said joining means to said non-woven material.
